(19) United States
(12) Patent Application Publication
(10) Pub. No.: US 2024/0285365 A1
(43) Pub. Date: Aug. 29, 2024

(54) STEREOTACTIC DEVICE AND METHOD FOR MANUFACTURING SUCH A STEREOTACTIC DEVICE

(71) Applicant: Steeve Chantrel, Nice (FR)

(72) Inventor: Steeve Chantrel, Nice (FR)

(21) Appl. No.: 18/573,393

(22) PCT Filed: Jul. 7, 2022
(86) PCT No.: PCT/EP2022/068934

§ 371 Date: Dec. 21, 2023

(30) Foreign Application Priority Data
Jul. 10, 2021 (FR) FR2107530

(51) Int. Cl. A61B 90/11; A61B 90/16; A61B 90/50

(52) U.S. Cl. CPC A61B 90/11; A61B 90/16; A61B 90/50

(57) ABSTRACT

The invention relates to a stereotactic device (10c) for an operation on the head of a patient (P), comprising: -at least one operating guide (71) configured to guide a surgical instrument, during the operation, to an operating zone; -at least one structure (5e) for positioning said at least one operating guide (71) relative to the skull of the patient (P); and -a dental support (3d) configured to anchor the positioning structure on the upper jaws of the patient (P); said at least one positioning structure (5e) being attached to the dental support (3d) as well as to the at least one operating guide (71).

### FIELD OF THE INVENTION

The present invention relates to a stereotactic device for surgery on the head of a patient, human or animal, as well as to the method of realizing the stereotactic device. Surgery on the head of a patient includes intracranial, ocular, inner ear, and sinus operations.

“Stereotaxy” refers to all methods allowing a three-dimensional mapping relative to the anatomy of a patient. More specifically, a stereotactic device enables an external reference point to be matched with one or more areas to be treated or preserved within the body of the patient. Typically, these areas only appear when medical imaging is performed.

The invention aims, in particular, to provide a guide for surgical tools with a reference point having a resolution and accuracy of better than a millimeter.

In this way, the invention has both therapeutic and surgical applications. Stereotactic surgery is used, for example, for the treatment of intracranial tumors, the placement of electrodes for Parkinson's disease, the prevention and treatment of strokes by dissolving clots or treating aneurysms, and the placement of cochlear implants.

Therapeutic applications involve the localized injection of therapeutic products, such as chemotherapy agents. For use on animals, stereotaxy also has applications in animal experimentation, notably for targeted injection of drugs.

### STATE OF THE ART

In animal experiments, rats are typically used by laboratories to test drugs. These drugs are injected intracranially into a target area of the brain of the rat. With current precision, approximately two-thirds of injections are carried out outside the target area. Consequently, these samples need to be discarded after post-mortem verification by dissection.

Generally speaking, when intervening in the brain or, more generally, in the head of a patient, whether a human or animal patient, great precision is required to avoid, as much as possible, any damage to the brain and other anatomical structures (eyes, inner ear, respiratory tract, etc.) of the patient.

Stereotactic devices are then used to guide physical or radiological interventions.

In the case of a physical operation, stereotactic devices are used to guide the movement of surgical instruments such as drills, biopsy needles, electrodes, cauterization devices, and so on.

To achieve this, these stereotactic devices incorporate an operating guide designed to guide and eventually hold the surgical instruments.

The stereotactic device most commonly used for physical operations is the stereotactic frame. As shown in FIG. 1, this frame (100) is screwed onto the skull (C) of the patient and serves as a reference point for the surgical instruments (105).

More specifically, FIG. 1 shows an example of stereotactic surgery in a simplified manner. In this example, a target operating zone is a tumor located at point P1. Starting from a first medical imaging, the surgeon determines the position of the operating zone, which is to say, the coordinates (x1, y1, z1) of the tumor on the medical image frame of reference, and then determines the optimal rectilinear axis of intervention A. This axis of intervention A passes through point P1 and a point P2 on the surface of skull C, known as the entry point. The medical imaging can also be used to determine the coordinates (x2, y2, z2) of point P2 in the medical imaging frame of reference. In the simplified example of FIG. 1, the face of the patient corresponds to the z axis of the medical imaging frame of reference so that aligning points P1 and P2 seems straightforward. In practice, aligning these points P1 and P2 requires a three-dimensional alignment.

In the context of an operation, without a stereotactic device, it is impossible for the surgeon to determine the required trajectory solely from the position of point P2, inasmuch as the operating zone is not visible when viewing the patient from outside. It is therefore advisable to obtain a point that is on the outside of the skull C and located on the axis of intervention A. In FIG. 1, this point bears reference sign P3. In this way, point P2 and point P3 enable surgical instruments 105 to be aligned along the axis of intervention A outside the skull C of the patient.

A Cartesian reference point is created around the skull of the patient to materialize point P3. Once established, this reference point can be used to unambiguously locate any point in the space surrounding the patient, both inside and outside skull C. To create the reference point, a rigid device, such as the frame 100 shown in FIG. 1, is literally screwed onto the skull C of the patient. The engagement of frame 100 to skull C without movement makes it possible to associate a coordinate system to the three-dimensional space surrounding the head of the patient.

After being screwed onto the skull C, An alignment operation is required for frame 100.

Indeed, the frame 100 is fastened on the outside of the patient, with no visibility of the anatomy of the brain. To guarantee the trajectory, it is imperative that the three points P1, P2 and P3 belong to the same reference point and have the same origin. This operation is achieved by radiological alignment, which requires additional imaging of the patient wearing the stereotactic frame 100. Without this radiological alignment, the device cannot be used.

Once the radiological alignment has been carried out, the Cartesian reference point materialized by the stereotaxic frame 100 maps the inside and outside of the skull C of the patient through a reliable coordinate system. The surgeon can then adjust the positioning of the surgical instruments 105 relative to the baseline of the frame 100, using an operating guide 103 fastened to the frame 100. This operating guide 103 takes the form of a cylindrical tube the central axis of which is positioned on the axis of intervention A.

The surgical instruments 105 can therefore be inserted into the operating guide to reach the skull C of the patient at point P2 and continue their course to point P1.

Thus, to perform stereotactic surgery using the frame 100 of FIG. 1, it is conventional to perform the following steps:
Performance of a first medical imaging;
Detection of an intracranial pathology in the first medical image;
Determination of the foreseen surgical act and the axis of intervention A;
Preparation and installation of frame 100 on the skull C of the patient;
Performance of a second medical imaging allowing the alignment of the operating guide 103 on the axis of intervention A; and
Use of surgical instruments 105 through the operating guide 103 to perform the foreseen surgical act.

The frame 100 enables the surgical instruments 105 to be held precisely in the operating guide 103, since the frame 100 has a direct power grip via screws 101 with the skull C of the patient. So, when the head of the patient moves, the frame 100 follows the movements of the head. A total immobilization of the patient is therefore not necessary between the various stages of preparation and installation of the frame 100, of alignment of the operating guide 103, and use of the surgical instruments 105 through the operating guide 103.

However, these operational steps are time-consuming inasmuch as before the actual operation can begin, the patient must be anesthetized for the installation of the frame 100, the patient must then be moved to the medical imaging device, and the medical imaging must be analyzed to align the operating guide 103. It follows that such an operation is complex to set up, inasmuch as the patient must be asleep for a long time, often several hours, to perform these various operations.

Moreover, in the case of a radiological intervention, which is to say, an operation using ionizing radiation, also known as radiotherapy, the treatments are carried out without any physical instrument and therefore without opening the skull C. For this type of radiological intervention, it is therefore not sought to guide the movements of a surgical instrument, but it is necessary to obtain precise positioning of the patient relative to the means of emission of the ionizing radiation, to ensure that all the radiation beams converge on the operating zone.

To ensure precise positioning of the patient, several approaches are possible, sometimes in combination, and other stereotactic means can be used.

A netting can be molded onto the face of the patient to immobilize the head of the patient on an operating table, either directly or indirectly. This device is sometimes supplemented by a fixation point at the maxillae of the patient. Indeed, the maxillae are integral with the skull, ensuring a reliable connection between a holding device fastened to the operating table and the skull of the patient.

Some radiosurgery units also use a maxillary device for spatial mapping. A dental splint is made to measure starting from the jaw of the patient.

This splint is connected to a device consisting of targets which are detectable in space by a sensor system. The patient is immobilized in a thermoformed netting, with the splint in place. In this way, the position of the sensors is detected in space to realign the focal point of the radiation-emitting means during a calibration stage.

However, these means of immobilizing the patient for radiological interventions do not allow physical operations to be carried out, as they do not allow surgical instruments to be guided.

A medical guidance device that does not overcome the difficulties of the aforementioned devices is also known from CN112603474A1, as it involves a column for attaching the device to the nose of the patient. Such an approach is imprecise inasmuch as the nose is reasonably elastic and cannot therefore guarantee a precise positioning. Similarly, EP2538856 A2 is a stereotactic device which requires medical imaging after fitting, with the disadvantages mentioned above. In addition to the disadvantage of multiple imaging, this method induces inaccuracy, inasmuch as a drilling template is manufactured as an input to the guide and then fastened to the guide. The combination of these constraints is a major factor in inaccuracy caused by successive additions of elements.

The technical task of the invention is therefore to provide a stereotactic device that limits the complexity of an operation using a stereotactic device, while at the same time guiding surgical instruments along a predetermined axis of intervention.

### DESCRIPTION OF THE INVENTION

To address this technical task, one aspect is to propose the use of a stereotactic guide connected to a dental support fastened to the maxilla of the patient.

Indeed, it has been observed that using the maxillae as a support for the stereotactic guide provides sufficient mechanical strength to support and/or guide the surgical instruments with great precision, so that it is not necessary to use a device screwed onto the skull of the patient.

The maxillary arch is, moreover, integral with the skull. An anchor on the maxilla therefore creates a three-dimensional reference point about the cranial anatomy of the patient. Unlike the position of a frame applied on the skull of the patient, the geometry of the maxillary arch can be digitally extracted from preoperative diagnostic imaging. It follows that the stereotactic device can be made to measure directly from the preoperative imaging, and it is no longer necessary to perform a second medical imaging procedure to achieve alignment of the stereotactic device.

Thus, according to a first aspect, a stereotactic device for operation on the head of a patient is presented, comprising:
at least one operating guide configured to guide, during the operation, a surgical instrument to an operating zone; and
at least one positioning structure relative to the skull of the patient of said at least one operating guide.

The device is characterized in that the stereotactic device also comprises a dental support configured to anchor the positioning structure to the maxillae of the patient, wherein said at least one positioning structure is fastened on the one hand to the dental support and on the other hand to the at least one operating guide.

The result is a made to measure stereotactic device, the dimensions of which can be determined using exclusively the first preoperative diagnostic imaging. During the operation, it is solely necessary to fasten the dental support to the maxillae of the patient to obtain a stereotactic device the operating guide of which is positioned directly on the axis of intervention.

It is possible to reduce the complexity of an intracranial operation, for which it is necessary to guide surgical instruments along a predetermined axis of intervention, by shortening the anesthesia time of the patient and by reducing the burden on medical staff.

The shape of the dental support and the positioning structure can vary.

According to one embodiment, the dental support comprises a splint made to measure relative to the maxillary arch of the patient, in such a way that the positioning device is hyperstatic when the splint is fitted to the teeth of the patient.

Indeed, the geometry of the maxillary arch, detected on preoperative diagnostic imaging, enables the creation of a made to measure dental splint, akin to the dental impression taken by dentists. This made to measure dental splint, once machined or printed, can be fitted on the maxillary teeth of the patient. Because of the U-shape of the dental arch and the shape of the teeth, the positioning of the splint is said to be hyperstatic, which is to say, with more stress than necessary to hold it in place. The hyperstatic nature of the connection ensures geometric continuity between the skull and the outside of the skull through the splint. This interface therefore guarantees an unambiguous mapping of the anatomical structures of the patient.

This splint can be fastened to the positioning structure in a unitary manner or be removable, for example, by means of a screw/nut system.

The use of removable fasteners enables the splint and positioning structure to be produced independently. It is then possible to use different materials to obtain different mechanical properties among these two elements. The splint can also be reused for several distinct operations associated with the same patient, with mechanical positioning structures materializing different axes of intervention or carrying distinct operating guides.

It is, moreover, possible to create a made to measure splint and to reuse an adjustable positioning structure for a plurality of patients.

To this end, the positioning structure may comprise at least two beams articulated to one another about an articulation, and means for adjusting the relative angular position of the two beams and for adjusting the distance between the articulation and the operating guide.

In this way, by adjusting the angular position of the articulation and the length of the two beams as a function of the settings made to measure for each patient, it is possible to reuse the same structure. This structure can, moreover, thus be conceived with very high strength using materials with specific mechanical performance, such as carbon, aluminum or high-density plastics.

As a variant, the positioning structure is a fixed one-piece structure, the dimensions of which are custom-determined using the position of the maxillae, the operating zone and the position of the operating guide. In contrast to an adjustable structure, a monoblock structure has the advantage of limiting positioning errors and improving the speed of installation on the patient, as it is no longer necessary to adjust the structure.

This made to measure monoblock structure can be produced with a 3D printer, using plastic materials selected for their mechanical performance and suitability for this type of printing. In addition to the position of the operating zone and the position of the operating guide, the shape of the face of the patient can also be established to determine the dimensions of the positioning structure.

Of course, the positioning structure may be adapted to guide a plurality of surgical instrument types, as a function of the needs of the operation. The operating guide may, moreover, comprise an end stop to limit the travel of the surgical instrument when the operating zone is reached. This end stop makes it possible to limit the travel of the instruments so that the instrument precisely reaches the operating zone, without exceeding it, when the end stop is reached.

When particularly heavy instruments or instruments applying high forces are required, it may be necessary to decouple the need for guidance from the need for support. To achieve this, the stereotactic device may comprise at least one power grip, configured to be fastened to an operating table on which the patient rests during the operation.

The strength of the retention of the dental anchorage may also be ensured by locking the jaw of the patient onto the dental anchorage in such a way that the lower teeth contribute in the fastening of the dental anchorage.

To this end, the stereotactic device may comprise a maxillary strap intended to grip the head of the patient by passing over the top of the head and the chin of the patient.

According to a second aspect, a method for manufacturing such a stereotactic device according to the first aspect is presented, wherein the method comprises the following steps:
creation of a three-dimensional image of the head of the patient;
determination of at least one operating zone in the three-dimensional image;
detection of the position and shape of the maxillae of the patient in the three-dimensional image;
determination of at least one entry point so as to reach the at least one operating zone with at least one predetermined axis of intervention;
determination of the position of at least one operating guide so that the central axis of said at least one operating guide is coaxial with the at least one predetermined axis of intervention connecting the at least one entry point and the at least one operating zone;
determination of the dimensions of the dental support as a function of the shape of the maxillae of the patient;
determination of the dimensions of the positioning structure as a function of the determined positions of the at least one operating guide and the maxillae; and
production of the stereotactic device on the basis of the determined dimensions.

This method enables a made to measure stereotactic device to be produced using a single step of three-dimensional intracranial imaging of the patient.

The various determination steps can be performed by a surgeon or an operator. For example, the operating zone is normally determined by a surgeon.

The entry point can also be selected by a surgeon. Alternatively, the determination of the at least one entry point on the skull of the patient can comprise a sub-step in which an artificial intelligence selects and suggests one or a plurality of possible entry points from which a surgeon can choose. In this way, an artificial intelligence trained on a large number of similar operations can suggest entry points previously used in similar cases.

Artificial intelligence can also be used to determine the shape and dimensions of the positioning structure to meet mechanical constraints. These mechanical constraints can also be estimated using artificial intelligence. As a variant, the shape of the structure can be selected from a set of predetermined shapes. These predetermined shapes then have adaptable dimensions that an algorithm can search as a function of the physiological measurements of the patient.

Image processing algorithms may likewise be implemented to determine the position of the operating guide, the dimensions of the dental support and/or the dimensions of an optional end stop in the operating guide.

Preferably, the manufacturing of the stereotactic device starting out from the determined dimensions comprises at least one step of 3D printing of the positioning structure, the operating guide and/or the dental support.

Another aspect of embodiments relates to a mounting method of a stereotactic device on the head of a patient, comprising the use of a device as previously introduced and its positioning on the head of the patient so that the positioning of the operating guide is ensured by the positioning of the support and of the positioning structure relative to the head of the patient, by means of the anchoring of the positioning structure on the maxillae of the patient.

### BRIEF DESCRIPTION OF THE FIGURES

The invention will be clearly understood from the following description, the details of which are given by way of example only, and will be developed in relation to the appended figures, in which identical reference signs refer to identical elements.

FIG. 1 shows a front view of a stereotactic device according to the prior art, screwed onto the skull of the patient.

FIG. 2 shows a perspective view of a head of the patient with a stereotactic device according to a first embodiment of the invention.

FIG. 3 shows a perspective view of the stereotactic device of FIG. 2.

FIG. 4 shows a flow chart containing the main steps in the method for manufacturing such a stereotactic device according to the invention.

FIG. 5 shows a medical imaging sub-step of the method of FIG. 4.

FIG. 6 shows a sub-step for determination of the axis of intervention of the method of FIG. 4.

FIG. 7 shows the spatial data extraction step for the manufacture of the stereotactic device according to the method of FIG. 4.

FIG. 8 shows a front view of a stereotactic device according to a second embodiment of the invention.

FIG. 9 shows a front view of the stereotactic device of FIG. 8, mounted on the maxillae of a patient.

FIG. 10 shows a front view of a stereotactic device according to a third embodiment of the invention.

FIG. 11 shows a perspective view of a stereotactic device according to a fourth embodiment of the invention.

FIG. 12 shows a perspective view of a stereotactic device according to a fifth embodiment of the invention.

FIG. 13 shows a top view of a stereotactic device according to a sixth embodiment of the invention; and

FIG. 14 shows a top view of a stereotactic device according to a seventh embodiment of the invention.

FIG. 15 shows another aspect of an embodiment of the invention, employing a press system.

FIG. 16 presents the assembly of the press system with the remainder of the stereotactic device.

FIG. 17 schematically illustrates how a splint and the press system work together.

FIG. 18 and FIG. 19 are respectively a front view and a side view of the head of a patient fitted with the stereotactic device of FIG. 15 through FIG. 17.

FIG. 20 is a partial view of the device according to the last embodiment.

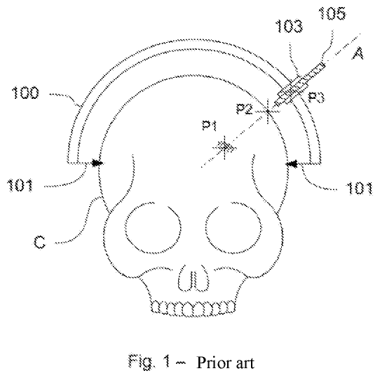

### DETAILED DESCRIPTION OF THE INVENTION

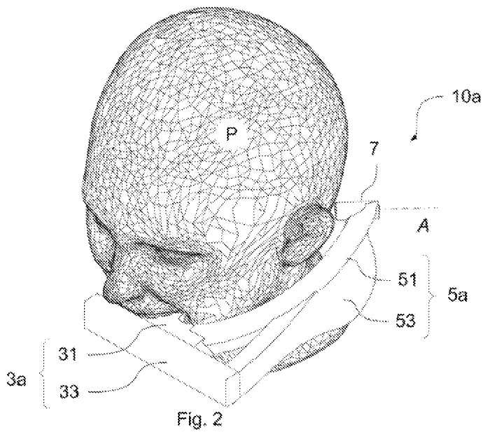

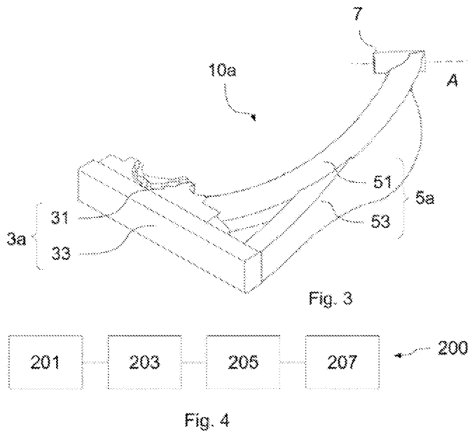

FIG. 2 shows the head of a patient P to which a stereotactic device 10a according to a first embodiment is fastened. This stereotactic device 10a is shown on its own in FIG. 3.

This stereotactic device 10a comprises a dental support 3a, a positioning structure 5a and an operating guide 7.

The dental support 3a presents a portion conforming to the maxillae 31 of the upper jaw of patient P, enabling the stereotactic device 10a to be anchored to a fixed point on the skull C of patient P. Indeed, the maxillae of the upper jaw are integral with the maxillary arch and therefore with the skull of the patient P. The portion conforming to the teeth is preferably in the form of a made to measure splint 31. The splint 31 is unitary with a base 33, located outside the mouth of patient P. In the example of FIG. 2 and FIG. 3, the base 33 takes the form of a horizontal bar extending laterally, when the dental support 3a is fastened on the patient P and the patient is standing with their head straight.

The operating guide 7 takes the form of a tube, the central axis of which coincides with an axis of intervention A. This operating guide 7 is used to guide surgical tools along the axis of intervention A.

The positioning structure 5a is fastened at one end to the dental support 3a and at the other end to the operating guide 7. The positioning structure 5a thus positions the operating guide 7 relative to the dental support 3a.

In FIG. 2, the operating guide 7 is positioned just behind the ear of patient P, which corresponds, for example, to the expected position of operating guide 7 for the placement of a cochlear implant.

The dental support 3a, the positioning structure 5a, and the operating guide 7 are made, for example, of a rigid plastic material (polyvinyl chloride PVC, polycarbonate PC, etc.). The stereotactic device 10a may, in particular, be manufactured in one piece; in which case the dental support 3a, the positioning structure 5a, and the operating guide 7 are all made from the same material.

As previously mentioned, the dental support 3a anchors the stereotactic device 10a relative to the maxillae of patient P, and thus relative to his skull, to which the maxillae are unitary. This anchoring is preferably hyperstatic, which ensures a power grip on the teeth by the dental support 3a, and thus that surgical tools can be applied to the operating guide 7 without dissociating the stereotactic device 10a from the skull of the patient P.

In this embodiment, the positioning structure 5a comprises a circular arc arm 51, connected at one end to the dental support 3a, in particular to its base 33, and at the other end to the operating guide 7. The positioning structure 5a, moreover, comprises a reinforcing bar 53, extending from the lateral end of the base 33 and joining the circular arc arm 51.

The shape of the positioning structure may vary according to the distance of the dental support 3a from the operating guide 7, the relative up/down or left/right orientation of the dental support 3a and the operating guide 7, the type of operation planned for the patient P, the type of instruments to be used, etc.

By manufacturing the stereotactic device 10a from a single piece, potential adjustment errors are avoided. In particular, the stereotactic device 10a can be manufactured using 3D printing, a manufacturing method that enables the manufacture of one-off or small-scale prototypes at low cost.

Alternatively, other machining methods can be used, in particular subtractive methods, for example, by means of a computer-aided (CAD or “computer-aided design”) milling machine. Through the use of a controlled milling or routing machines, it is thus possible to obtain the desired shape for the stereotactic device 10a.

Combinations of additive and subtractive machining methods can also be used: it is possible to mold or print a rough template (additive phase) and to then precisely machine the fine structure of the stereotactic device 10a by means of a computer-aided machine (milling or routering).

FIG. 4 through FIG. 9 illustrate the manufacturing method of a stereotactic device according to the invention.

FIG. 4 is a flow chart schematically illustrating the steps in the method 200 for producing a stereotactic device 10b according to a second embodiment.

The first step 201 consists in determining the position of the target zone, which is to say, point P1, in a three-dimensional medical imaging reference point of the head of the patient P. This point P1 can be identified by coordinates x1, y1, z1 in a Cartesian system. According to a variant, the mapping can be performed in a spherical system.

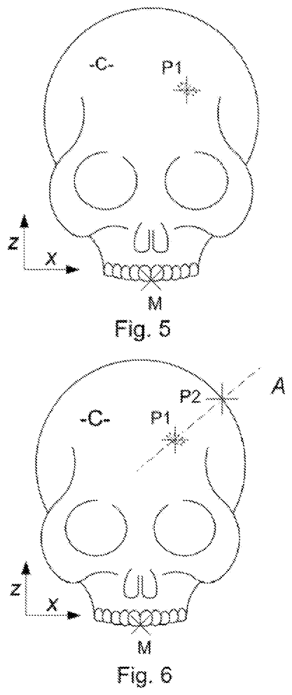

In a Cartesian x, y, z system, the head of the patient P is represented schematically by their skull C. The point M, with coordinates xM, yM, zM, represents the position of the maxillae. More specifically, a set of points M is sought to determine the contours of the teeth for the manufacture of the dental support 3b, as shown in FIG. 8. This first step is schematically illustrated in FIG. 5.

During the second step 203, the entry point P2 and/or the axis of intervention A are determined. The entry point P2 and the axis of intervention A are, in particular, chosen by the surgeon to avoid vital or sensitive areas (blood vessels, nerves, etc.). This second step is schematically illustrated in FIG. 6.

In particular, depending on the type of operation and of the patient data, an artificial intelligence can pre-select several entry points P2, amongst which the surgeon chooses the one they consider most appropriate. The P2 point is then mapped by its x2, y2, z2 coordinates in the same reference point.

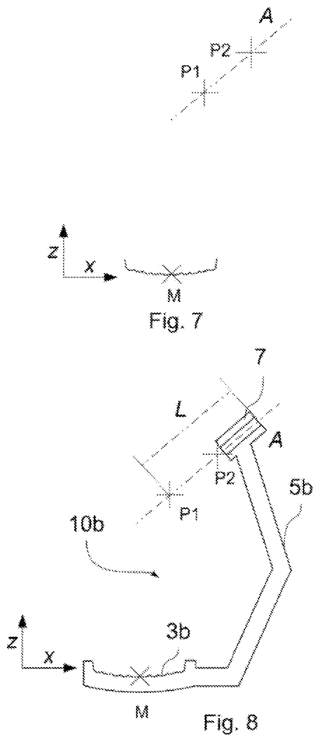

The third step 205 is the dimensioning of the stereotactic device 10b based on the positions of points P1, P2, M and the contour of the teeth around point M, then extracted from the single three-dimensional image of the head of the patient P. These elements are shown schematically in FIG. 7.

In particular, depending on these data, the type of operation and the surgical tools used, different shapes can be proposed for the positioning structure 5b. Once again, artificial intelligence can pre-select several shapes, from which the surgeon will select the one they consider most suitable.

The fourth step 207 then consists in manufacturing the stereotactic device 10b, for example, by means of 3D printing. This step is illustrated schematically in FIG. 8, which shows the stereotactic device 10b thus obtained.

Depending on the length of the surgical instruments to be used, the shape of the operating guide 7 can be parameterized to form an end stop at a predetermined distance L from the target zone P1. This end stop prevents the surgical instruments from being pushed in too far or too little. Alternatively, the end stop can be implemented by addition to the overall tubular shape of the operating guide.

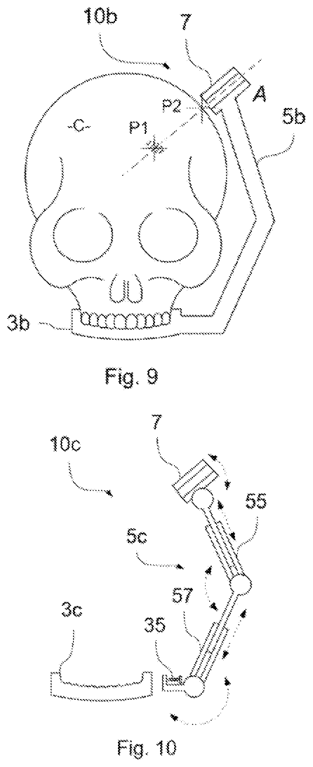

In the embodiment of FIG. 9, the stereotactic device 10b is placed on the patient P by means of the dental support 3b attached to the teeth.

FIG. 10 shows an alternative embodiment of a partially reusable stereotactic device 10c.

In this embodiment, the dental support 3c is detachable from the positioning structure 5c, and the positioning structure 5c is adjustable.

In particular, positioning structure 5c comprises two beams 55, 57, articulated to each other and relative to dental support 3c. Beams 55, 57 are telescopic, and thus present an adjustable length.

More specifically, the beams 55, 57 are articulated to each other by a pivot or ball-and-socket joint. In this way, each beam 55, 57 is attached to the pivot or ball-and-socket joint on the one hand, and to the dental support 3c or operating guide 7 on the other, preferably via pivot or ball-and-socket joints.

With such a stereotactic device 10c, the third dimensioning step 205 produces an adjustment of the position of beams 55, 57 relative to each other and in relation to the dental support 3c, as well as of the orientation of the operating guide 7 at the end of beams 55, 57. To this end, the articulations and telescopic beams 55, 57 may comprise a scale, such as a vernier scale, and means for locking them in position.

As a variant, a positioning structure can comprise three beams or more without changing the invention. The beams can be aligned or laid out in a Y-shape, with two or more operating guides 7 at the free ends.

Such positioning structures can, in particular, be in kits, with the dimensioning step 205 providing the references and assembly order of the articulated beams, articulations, and operating guides to obtain the appropriate stereotactic device.

The manufacturing step 207 of the stereotactic device then consists in an assembly of the various elements according to the references and assembly order provided in the dimensioning step 205.

The dental support 3c is, for example, removably and interchangeably attached by screws 35 to one end of the positioning structure 5c. The operating guide 7 can also be attached by screws or by a form-fit.

In this way, it is possible to use the positioning structure 5c with different dental supports 3c. As the dental support 3c is the individualized portion of the stereotactic device 10c, it is consequently possible to reuse the adjustable positioning structure 5c for several patients P, or to use different positioning structures 5c of different types or that are pre-adjusted to different settings for the same patient P, on which the dental support 3c then remains in place.

The positioning structure 5c can therefore be made of metal, with precise machining because it can be reused for several operations. A suitable metal is, for example, so-called “surgical” stainless steel or aluminum.

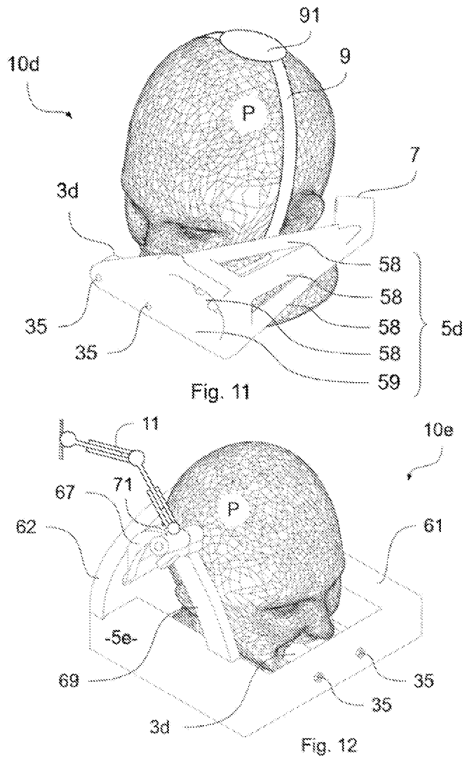

FIG. 11 shows an alternative embodiment of a stereotactic device 10d according to the invention. This stereotactic device 10d comprises a positioning structure 5d attached to the dental support 3d by screws 35, and comprising four arms 58. The arms 58 extend from a base 59 of the positioning structure 5d, bearing the screws 35, the base 59 extending on the side of the head of the patient P. The arms 58 extend from the lateral extension side of the base 59 and meet at the operating guide 7.

The arms 58 thus form a pyramidal structure, wherein the operating guide 7 is at the apex, and wherein the base 59 of the positioning structure 5d forms the base. By manufacturing a dental support 3d and a positioning structure 5d connected by screws 35, it is possible to manufacture these different elements of different materials, each having different mechanical properties (strength, weight, cost and machining properties).

The stereotactic device 10d moreover comprises a maxillary strap 9, which encircles the head of the patient P, passing over the top of the head (arch or sinciput) and the chin. The tightening of this maxillary strap 9 enables the maxillary support 3d to reinforce its power grip on the teeth by compressing the mandible of patient P, imitating a biting force. The maxillary strap 9 comprises, for example, an elastic strap, and/or tightening means such as a buckle or ratchet system.

The maxillary strap 9 likewise comprises a cap 91 that is concave in shape and arranged on the top of the skull so as to match its shape, and correspondingly a chin strap (not shown) at the chin of the patient P.

The stereotactic device 10d can therefore be used with heavier surgical tools that require greater effort to guide them.

FIG. 12 shows another embodiment of a stereotactic device 10e according to the invention, worn by a patient P whose head is shown in three-quarter view. The positioning structure 5e of the stereotactic device 10e of FIG. 12 comprises a frame 61, which runs around the head of the patient P. This frame 61 may be substantially parallelepipedic and located at the height of the mouth in the horizontal plane, when the patient P is standing with head straight.

At the rear of the head of the patient, towards the occiput, the frame 61 may comprise a raised wall 62, with a concave recess 63 that follows the shape of the occiput of patient P.

To ensure that it is anchored to the skull of the patient P, the frame 61 comprises holes that cooperate with screws 35 to fasten it to the dental support 3d.

The operating guide 71 is borne by two arch-shaped arms 67, 69. A first arm 67 is fastened to the raised wall 62 of the frame 61, whereas the second arm 69 is fastened directly to one of the bars forming the frame 61.

For ease of installation, the frame 61 can be machined in several detachable parts, for example, the raised wall 62 can be removable and fastened by screwing or by a form-fit.

The operating guide 71 shown presents an end stop 75 which extends its tubular shape. Such an operating guide 75 typically corresponds to an operation where the target zone P1 is shallow relative to the surface of the skull C.

In the embodiment of FIG. 12, the operating guide 71 is connected to a power grip, in the form of an articulated arm 11, connected on the one hand to the operating table (not shown) and on the other hand to the operating guide 71. The articulated arm 11 comprises locking means, so that large forces are required to change its configuration when it is locked.

The anchoring obtained by the power grip in the form of an articulated arm 11 on the frame 61 enables to once again use surgical tools exerting potentially high forces on the operating guide 71.

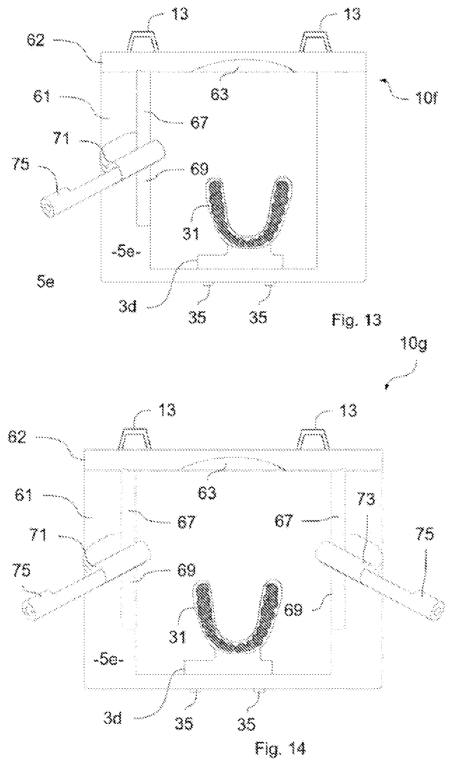

FIG. 13 is a top view of a stereotactic device 10f that is similar to that of FIG. 12.

The stereotactic device 10f of FIG. 13 likewise comprises, in particular, a frame 61, with a raised wall 62 provided with a recess 63 matching the shape of the occiput of the patient P (not shown in FIG. 13).

On the other hand, the anchoring of the stereotactic device 10f is ensured by fasteners 13, arranged at the rear of the raised wall 62. These fasteners 13 are solid loops or arches which, for example, snap into a corresponding anchoring mechanism on the operating table (not shown), upon which the patient P rests during the operation.

Indeed, the rear surface of the raised wall 62, at the occiput of the patient P, is typically the surface facing the surface of the operating table when the patient P is lying on their back.

If the operation requires the patient P to lie on their side, the fasteners 13 may, in particular, be arranged on the corresponding side of frame 61.

FIG. 14 shows the possibility of using several operating guides 71, 73 with the same stereotactic device 1.

In this way, the stereotactic device 10g of FIG. 14 is similar to that of FIG. 12 and FIG. 13. The stereotactic device 10g of FIG. 14 also comprises a frame 61, with a raised wall 62 provided with a recess 63 that follows the shape of the occiput of the patient P.

The stereotactic device 10g of FIG. 14, on the other hand, comprises two operating guides 71, 73, arranged on each side of the frame 61, in a substantially symmetrical fashion, with each of the arms 67, 69 connecting them to the frame 61.

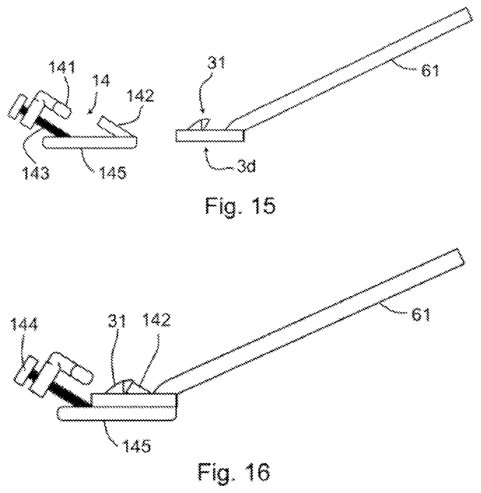

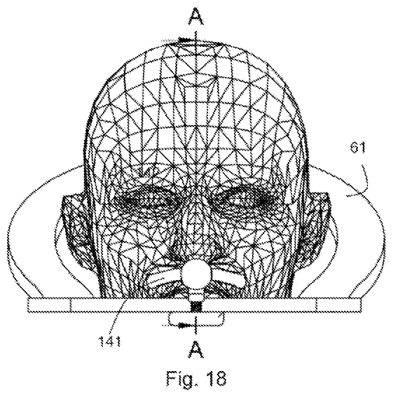

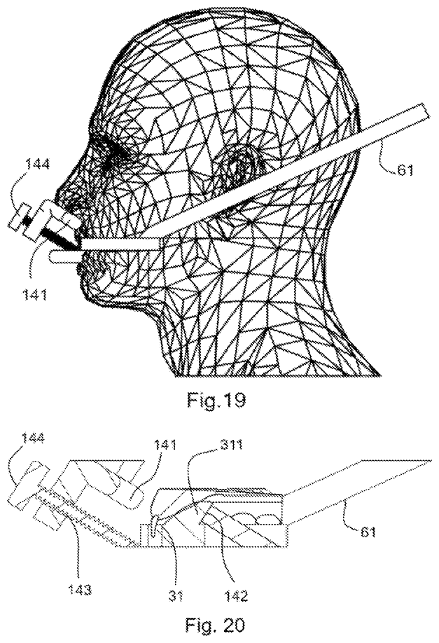

FIG. 15 through FIG. 20 show a further embodiment.

According to this example, the dental support 3d comprises a press system 14 having a first part 141 provided with a bearing surface adapted to be applied, without screwing, to the face of the patient P, preferably between the nose and the upper lip, and a second part 142 provided with a counter-bearing surface adapted to be applied to a portion of the splint 31 at the rear of the maxillary arch of patient P, wherein the first part and the second part are configured to be drawn closer together so as to exert pressure on the splint 31 on both sides of the maxillary arch of the patient P.

If the patient is an animal, the pressure is exerted on the muzzle, preferably above the snout, between the snout and the eyes and along the muzzle; this may be the area known as the bridge of the nose in dogs.

FIG. 15 shows a side view of such a press system 14 with a base 145, for example, directed in a plane that may be oriented perpendicular to the sagittal plane of the patient P. The base 145 acts as a support to a first part 141 and a second part 142, producing a clamping about the upper maxillary arch of the patient on opposite sides of the splint 31. Preferably, this clamping is performed with opposing pressures from parts 141, 142 which pressures are directed in the same plane or in parallel planes that are not far apart from one another, for example, by less than 10 mm. Preferably, the average direction of pressure of the first part 141 and the second part 142 is directed along the sagittal plane of the patient.

To implement the vice principle, parts 141, 142 can be drawn together (or moved apart to release the device). The press system advantageously comprises a helical slide configured to move one of either the first part or the second part in translatory motion in relation to the other of either the first part or the second part.

In the example shown, to facilitate handling, it is the first part 141, which is accessible from outside the mouth as it is intended to be applied to the face of the head of the patient, whose movement is controlled. The second part 142 is fixed according to this non-limiting example. According to FIG. 15, the mobility of the first part 141 is ensured by a helical slide 143 in the form of a worm screw rotatably mounted on the base 145 so as to be movable in rotation with actuation via a screwing head 144. The first part 141 is mounted about the screw so as to translate in the longitudinal direction of the screw when the screwing head 144 is actuated.

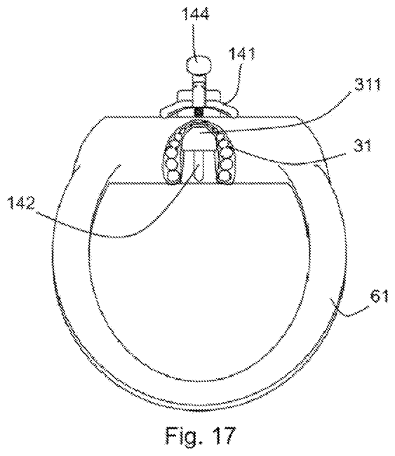

The second part 142 may take the form of a rod, preferably oriented in the sagittal plane and preferably inclined relative to the plane of the splint, for example, by at least 10°. The distal end of the rod forms the counter-pressure surface. This surface rests on the rear face of splint 31, itself behind the upper maxillary arch. As shown particularly in FIG. 17, this portion of the splint can form a protrusion 311 with more material than the rest of the rear area of the splint. The zone 311 offers a volume of material allowing to distribute the pressure stemming from the counter-pressure surface, which surface may be smaller in size. In this way, zone 311 forms a buffer element and an element for distribution of the pressure exerted by press system 14. The zone 311 of the splint can be formed separately from the rest of the splint 31, and attached by any means, such as clipping.

FIG. 20 shows the stereotactic device in a partial manner and in cross-section in a zone of cooperation between the press system 14 and the splint 31, about the upper maxillary arch. The teeth are retained by the splint by the hyperstatic support that is created on their surface. This retention is here supplemented by the press 14, which forms an additional vice for the upper maxillary arch; in the example shown, via the counter-pressure zone 142, the projection 311 of the splint is applied to a portion that is in front of the teeth of the maxillary arch and, in counterpoint, via the support zone 141, the press exercises a thrust at the upper lip of the patient. It is understood that the anchoring is optimized inasmuch as it is completed and distributed in this area of the face of the patient.

The bearing surface of the first part preferably has an arched shape. This arrangement is more particularly visible in FIG. 17 through FIG. 19. This enables the contour of the face to be followed in the application area of the first part 141. The first part is in effect in the form of an arc, following the contour of the outer surface of the maxillary arch.

According to FIG. 16, the press system 14 is assembled to the remainder of the device, preferably by fastening of the base 145 at a base of the dental support 3d.

As in the embodiment of FIG. 12 and FIG. 13, the dental support may itself receive a positioning structure, such as the one shown with frame 61 in FIG. 15 through FIG. 20, wherein this frame may completely surround the head of the patient. The device thus formed is, preferably, symmetrical in the sagittal plane of the patient, so as to balance out the weight of the assembly relative to the anchorage provided by the splint 31 and, optionally, supplemented by the press system 14. In this example, the frame 61 is rounded, in the overall form of a ring.

The first and second parts may be configured to exert pressure on the splint 31 along a plane inclined by at least 10° relative to a plane of the splint. This makes it possible to distribute the anchoring directions of the splint relative to the maxillae.

In one option, the clamping by the press system is carried out by two opposing pressures transmitted to the upper maxillary arch. Alternatively, these pressures have a mean direction located above the teeth, at the height of the maxillary bone.

Other embodiments may provide that the same arm may bear a plurality of operating guides if their positions are sufficiently close, or instead a single-piece operating guide may comprise a plurality of holes along different axes of intervention A, if said axes of intervention A are sufficiently close.

Stereotactic devices 10a-10g according to the invention are thus available in numerous variants, adapted to different operations on the head of a patient P, such as biopsies, intracranial injections, placement of electrodes or implants such as cochlear implants, and so on.

All these variants, however, have the advantage that they can be performed using a single three-dimensional medical imaging image, typically the one used for the precise diagnosis of patient P. The medical imaging steps are thus limited for patient P. The dental support 3a-3d and said at least one positioning structure 5a-5e are determined to ensure the positioning of said operating guide 7, 71, 73 on an axis of intervention A determined from at least one preoperative imaging, which is to say, without performing a second medical imaging to obtain alignment of the stereotactic device.

The implementation of the stereotactic devices according to the invention is moreover simple and quick, and does not require the tightening of a plurality of screws against the skull of the patient P, as is the case with stereotactic frames in the state of the art. These screws are, moreover, prone to loosening or to be under-tightened, which can bring about slippage of the frame and thus of a shift of the axis of intervention A relative to the target zone P1. The success of the operation may then be compromised.

The stereotactic devices 10a-10g according to the invention thus provide enhanced safety during the operations.

Any aspect of any of the above-described embodiments may be combined with any other compatible aspect of any one of the other embodiments.

1. A stereotactic device, for operation on the head of a patient, comprising:
at least one operating guide configured to guide, during the operation, a surgical instrument to an operating zone; and
at least one positioning structure relative to the skull of the patient of said at least one operating guide;
the stereotactic device comprising a dental support configured to anchor the positioning structure to the maxillae of the patient such that the dental support ensures the positioning of the stereotactic device relative to the head of the patient;
wherein said at least one positioning structure is fastened on the one hand to the dental support and on the other hand to the at least one operating guide, wherein the dental support and said at least one positioning structure being determined to ensure the positioning of said operating guide on an axis of intervention determined from at least one preoperative image.

2. Stereotactic device according to claim 1, wherein the positioning structure is a fixed one-piece structure the dimensions of which are custom-determined using the position of the maxillae, the operating zone and the position of the operating guide.

3. Stereotactic device according to claim 1, wherein the positioning structure comprises at least two beams articulated to one another about an articulation, and means for adjusting the relative angular position of the two beams and for adjusting the distance between the articulation and the operating guide.

4. Stereotactic device according to claim 1, wherein the dental support comprises a splint made to measure relative to the maxillary arch of the patient in such a way that the positioning structure is hyperstatic when the splint is fitted to the teeth of the patient.

5. Stereotactic device according to claim 4, wherein the splint is attached to the positioning structure by removable fastening means.

6. Stereotactic device according to claim 4, wherein the dental support comprises a press system having a first part provided with a bearing surface adapted to be applied to the face of the patient and a second part provided with a counter-bearing surface adapted to be applied to a portion of the splint at the rear of the maxillary arch of the patient, wherein the first part and the second part are configured to be drawn closer together so as to exert pressure on the splint on both side of the maxillary arch of the patient.

7. Stereotactic device according to claim 6, wherein the bearing surface of the first part has an arched shape.

8. Stereotactic device according to claim 6, wherein the press system comprises a helical slide configured to move one of either the first part or the second part in translatory motion in relation to the other of either the first part or the second part.

9. Stereotactic device according to claim 6, wherein the first part and the second part are configured to exert pressure on the splint along a plane inclined by at least 10° relative to a plane of the splint.

10. Device according to claim 6, wherein the bearing surface of the first part is configured to be applicable between the nose and upper lip of the patient.

11. Device according to claim 6, wherein the bearing surface of the first part is configured to be applicable on the muzzle.

12. Stereotactic device according to claim 1, wherein the operating guide comprises a stop to limit the travel of the surgical instrument when the operating zone is reached.

13. Stereotactic device according to claim 1, wherein the stereotactic device also comprises at least one power grip, configured to be fastened to an operating table on which the patient rests during the operation.

14. Stereotactic device according to claim 1, comprising a maxillary strap intended to grip the head of the patient, by passing over the top of the head and the chin of the patient.

15. A method for manufacturing a stereotactic device according to claim 1, wherein the method comprises the following steps:
creating a three-dimensional image of the head of the patient;
determining at least one operating zone in the three-dimensional image;
detecting the position and shape of the maxillae of the patient in the three-dimensional image;
determining at least one entry point so as to reach the at least one operating zone with at least one predetermined axis of intervention;
determining the position of at least one operating guide so that the central axis of said at least one operating guide is coaxial with the at least one predetermined axis of intervention connecting the at least one entry point and the at least one operating zone;
determining the dimensions of the dental support as a function of the shape of the maxillae of the patient;
determining the dimensions of the positioning structure as a function of the determined positions of the at least one operating guide and the maxillae; and
producing the stereotactic device on the basis of the determined dimensions.

16. Method according to claim 15, wherein the determination of the at least one entry point on the head of the patient comprises a sub-step in which an artificial intelligence selects and suggests one or a plurality of possible entry points from which a surgeon can choose.

17. Manufacturing method according to claim 15, wherein the manufacturing of the stereotactic device starting out from the determined dimensions comprises at least one step of 3D printing of the positioning structure, the operating guide, and/or the dental support.

18. Device according to claim 11, where the bearing surface of the first part is configured to be applicable between the snout and the eyes of the patient when said patient is an animal.
